(19) United States
(12) Patent Application Publication
(10) Pub. No.: US 2009/0063259 A1
(43) Pub. Date: Mar. 5, 2009

(54) INFORMATION SYSTEM FOR BIOLOGICAL AND LIFE SCIENCES RESEARCH

(76) Inventors: Ramin Cyrus, Vienna, VA (US); Matteo Di Tommaso, Gaithersburg, MD (US); Anthony R. Kerlavage, Montomery Village, MD (US); Charles B. Lawrence, Rockville, MD (US)

Correspondence Address: HARNESS, DICKEY & PIERCE, P.L.C., P.O. BOX 828, BLOOMFIELD HILLS, MI, 48303, US

(21) Appl. No.: 12/207,909

(22) Filed: Sep. 10, 2008

Related U.S. Application Data
(62) Division of application No. 10/643,204, filed on Aug. 15, 2003.
(60) Provisional application No. 60/495,506, filed on Aug. 15, 2003.

(51) Int. Cl. G06Q 30/00; G06F 17/30; G06Q 10/00; G06Q 50/00

(52) U.S. Cl. 705/11; 707/102; 705/26; 707/E17.002

(57) ABSTRACT

An online life science research environment and virtual community with a focus on design and analysis of biological experiments includes a life sciences laboratory system employing at least one networked computer system that defines a virtual research environment. Users access the system through a portal associated with the networked computer system(s). The virtual research environment has a data coupling mechanism by which the user designates a set of user-specified data for bioinformatics processing. A processor(s) associated with the networked computer system(s) performs bioinformatics services upon the user-specified data. In one embodiment, the data coupling mechanism enables transfer of the user-specified data to a memory space that is mediated or accessed by the processor performing the bioinformatics processing. Users may thus exploit bioinformatics processing resources that are not deployed on users' local computer environments, and to store and organize information relating to life sciences research in a secure, online workspace.

### FIELD

The present disclosure relates generally to information systems for biological and life sciences research. More particularly, the disclosure relates to a network-based virtual research laboratory and collaboration portal with which biological and life sciences research may be more efficiently conducted.

### BACKGROUND

Humanity passed a significant milestone in unraveling the mysteries of life on Jun. 26, 2000, when Dr. Craig Venter and Dr. Francis Collins stood proudly beside President Clinton to announce that the code of the human genome had been cracked, nearly two years ahead of schedule. In President Clinton's words, “Today, we are learning the language in which God created life.” His meaning: research scientists have now identified and recorded, in computer database form, the some 3 billion base pairs that comprise the entire human genome. This was a stunning achievement, but it is only the beginning.

According to recent estimates there are 30,000 to 40,000 genes in the human genome. While the identity and sequences of the 3 billion base pairs has now been worked out, little is yet known about which of these base sequences correspond to the 30,000 to 40,000 genes. Similarly, little is yet known about which of these base sequences are responsible for which proteins and bodily functions, or which of these base sequences are implicated in treating disease. In short, there is much to learn.

In practical effect, the decoding and storing of the human genome in a computer database has changed biology from an information gathering science into an information processing science. Computer scientists have joined the ranks of the laboratory scientists to spawn a new field, called computational biology—the application of quantitative analytical techniques in modeling biological systems. Much of the effort in this new field has been devoted to the science of using information to understand biology. Computer scientists call this science, bioinformatics.

To the bioinformatics computer scientist, the human genome represents a vast data-mining project that holds profound promise to cure disease and prolong our lives. The current approach to data-mining involves applying statistical methods and pattern recognition algorithms upon the genome database to make predictions about the information that is locked in our DNA. The nature of the problem is such that computer scientists must perform these analytical tasks without a complete understanding of where the biological data comes from or what it means.

Moreover, the bioinformatics field is still in its infancy. Currently, many life sciences researchers are struggling to learn how to employ computational tools in their work. Unfortunately, many of the computational tools require quite sophisticated knowledge of computer science and statistical mathematics, not to mention vast computational resources. This has placed many of the more promising analytical techniques off-limits to all but the largest research companies and institutions. For humanity's sake, this is quite unfortunate, because it squanders the full potential of humanity's creative minds. These are the creative minds working, without great funding, throughout the many small university and private research laboratories around the world-creative minds which would be capable of making significant, life-improving discoveries if empowered with the right tools.

Some recently developed tools and techniques related to these endeavors are discussed in the following patent applications, each assigned to the assignee of the present invention: U.S. Provisional Application No. 60/386,296, entitled Informatics System Architecture, and filed Jun. 4, 2002; U.S. Provisional Application No. 60/411,574, entitled Integration Instructions for Informatics Systems Architecture, and filed Sep. 16, 2002; U.S. application Ser. No. 10/455,262, entitled System and Method for Open Control and Monitoring of Biological Instruments, and filed Jun. 4, 2003; U.S. application Ser. No. 10/455,264, entitled System and Method for Discovery of Biological Instruments, and filed Jun. 4, 2003; U.S. application Ser. No. 10/455,579, entitled System and Method for Providing a Standardized State Interface for Instrumentation, and filed Jun. 4, 2003; U.S. application Ser. No. 10/455,263, entitled System and Method for Generating User Interfaces for Different Instrument Types, and filed Jun. 4, 2003; U.S. application Ser. No. 10/334,793, entitled Method for Placing, Accepting and Filling Orders for Products and Services, and filed Jan. 2, 2003; PCT Application No. US0234599, entitled Method for Operating a Computer and/or Computer Network to Distribute Biotechnology Products, and filed Oct. 30, 2002; U.S. Provisional Application No. 60/431,879, entitled A Browsable Database for Biological Use, and filed Dec. 19, 2002; U.S. Provisional Application No. 60/433,421, entitled Methods for Identifying Orthologous Genomic Regions Between Two or More Species, and filed Dec. 13, 2002; and U.S. Provisional Application No. 60/466,310, entitled Methodology and Graphical User Interface to Visualize Genomic Information, and filed Apr. 28, 2003. The disclosures of each of the aforementioned patent applications are incorporated herein by reference.

### SUMMARY

The present system provides a life sciences laboratory system employing at least one networked computer system that defines a virtual research environment. Users access the system through a portal associated with the networked computer system(s). The virtual research environment has a data coupling mechanism by which the user designates a set of user-specified data for bioinformatics processing. At least one processor associated with the networked computer system(s) performs bioinformatics services upon the user-specified data. In one embodiment, the data coupling mechanism enables transfer of the user-specified data to a memory space that is mediated or accessed by the processor performing the bioinformatics processing. This embodiment allows users to exploit bioinformatics processing resources that are not deployed on users' local computer environments, and to store and organize information relating to life sciences research in a secure, online workspace.

In another embodiment, the data coupling mechanism enables transfer of bioinformatics processing routines to a memory space that is mediated or accessed by the processor that locally accesses the user-specified data. This embodiment allows users to perform bioinformatics processing operations locally, without security concerns that others may be able to access their user-specified data.

According to a further aspect, a virtual community system is provided to facilitate collaboration and sharing of life sciences information. At least one networked computer system defines a virtual community that is accessible by a plurality of users. The virtual community provides information linking services whereby users may provide references to life sciences information. The system includes an index service provider, associated with the virtual community, that coordinates the provided references to life sciences information. Coordination is through an information architecture that defines hierarchical levels and defines links among related information across the hierarchical levels.

In one embodiment, the index service provider uses an indexing or cataloging system, based on the genome itself, that establishes a unified indexing schema or coordinate system. The indexing system provides a common reference system by which otherwise disparate blocks of information can be associated with one another.

In yet another aspect, the system provides a life sciences network portal system employing at least one networked computer system that defines the portal. Users may access the networked computer system through the portal to conduct life sciences research. The portal system includes a workflow system that is operable to allow a user to prescribe and track the performance of a series of steps associated with that user's life sciences research.

The system includes a data store of life sciences information accessible through the portal, as well as a product specifying system that identifies offered products useful in connection with performing the series of steps. An indexing mechanism associated with the networked computer system mediates relationships among the workflow system, the data store of life sciences information and the product specifying system.

According to a further aspect, the life sciences laboratory system employs at least one networked computer system that defines a virtual research environment accessible to a user through a portal associated with the networked computer system. The computer system is configured according to a framework that defines a common communication interface to a plurality of different life sciences laboratory equipment. The framework further defines a virtual laboratory equipment interface presented through the portal, whereby the user may interact with selected ones of the plurality of different life sciences laboratory equipment.

The framework allows users to establish working links between plural different life components of, otherwise incompatible, sciences equipment that may be located anywhere in the world.

Still further, a life sciences workflow management system employing at least one networked computer system is configured to provide a workflow interface to a user through a portal. The workflow interface is operable to allow a user to prescribe and track the performance of a series of steps associated with life sciences research. The system employs a data store associated with the networked computer system into which the user stores a set of user-specified data for bioinformatics processing. At least one processor associated with the networked computer system is configured to perform bioinformatics processing upon the user-specified data. The workflow interface has a user interaction mechanism whereby the user can manipulate user-specified data stored in the data store and whereby the user can control the performance of the bioinformatics processing.

Further areas of applicability of the present system will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

The present system will become more fully understood from the detailed description and the accompanying drawings, wherein:

FIG. 1 is a system diagram illustrating the information system;

FIG. 2 is a data structure diagram illustrating the presently preferred indexing technique employed by the system;

FIG. 3 is a further data structure diagram illustrating further aspects of the indexing system;

FIG. 4 is an information hierarchy diagram, illustrating how the information system utilizes and processes information;

FIG. 5 is an exemplary website implementation of an information system;

FIG. 6 is a use case example, based on the website implementation of FIG. 5;

FIG. 7A is a first example of a work flow, implemented using the information system;

FIG. 7B is a second example of a work flow implemented using the information system;

FIG. 8 is another example of a workflow implemented usig the information system;

FIG. 9 is a hardware architecture diagram illustrating an exemplary implementation of the information system;

FIGS. 10A and 10B is a web navigation diagram of an exemplary portal implementation;

FIGS. 11-20 are web page diagrams illustrating an exemplary web-based portal implementation of the information system;

FIG. 21 is a block diagram illustrating a workflow framework design tool of the information system.

### DETAILED DESCRIPTION OF VARIOUS EMBODIMENTS

The following description of the various embodiment(s) is merely exemplary in nature and is in no way intended to limit the invention, its application, or uses.

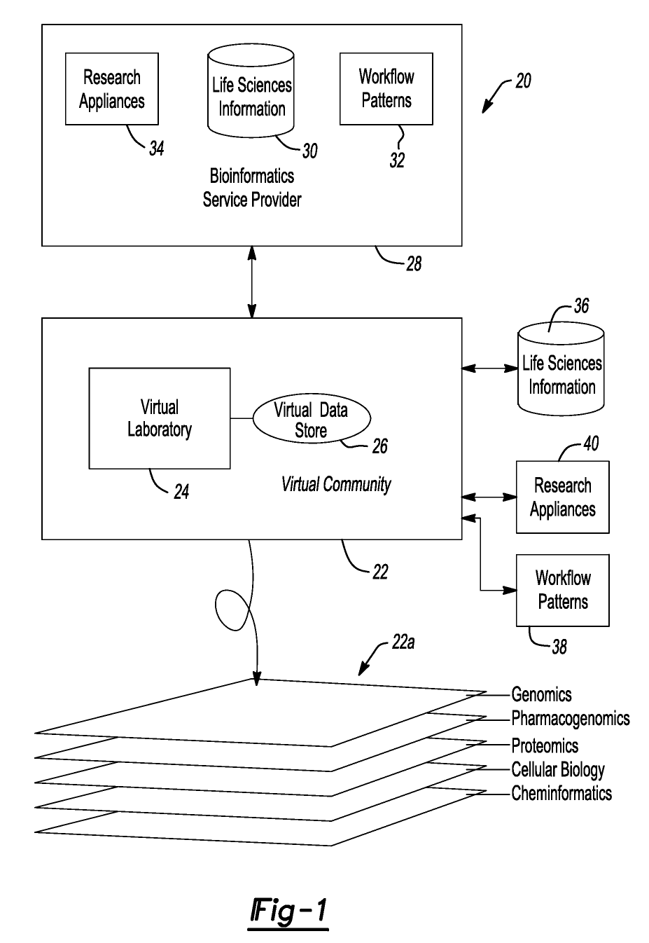

An information system is illustrated diagrammatically in FIG. 1. The information system 20 is preferably implemented using a networked computer system, such as the Internet, to define a virtual community 22. As will be more fully explained, the virtual community defines a virtual workspace having at least one, and preferably many, virtual laboratories 24, each having an associated virtual data store 26. The virtual laboratory represents a workplace in the virtual community 22 where the biological or life sciences researcher can conduct in silico experiments, upload, download and analyze data, design and perform experiments using system-generated work flows, conduct information research and share information with others in the virtual community.

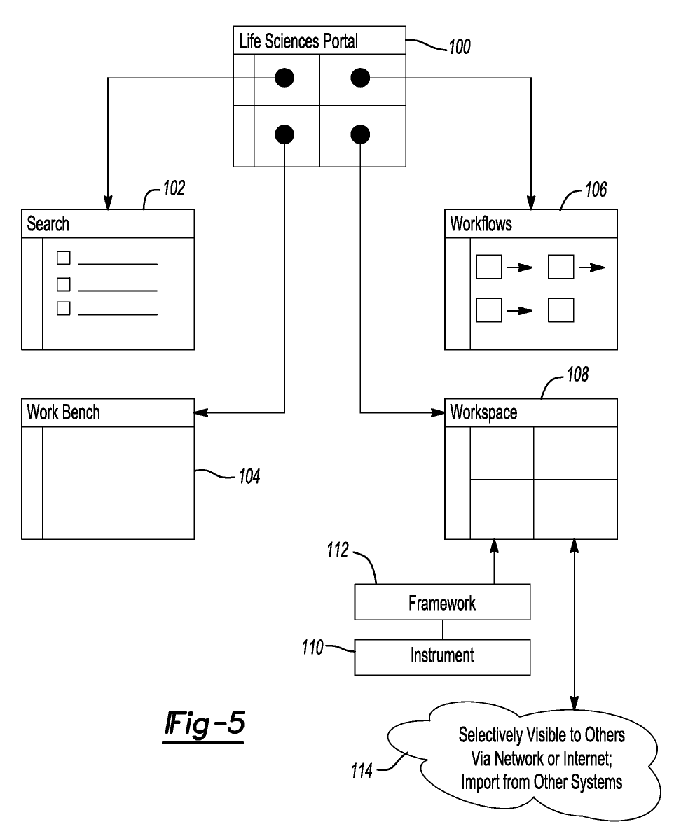

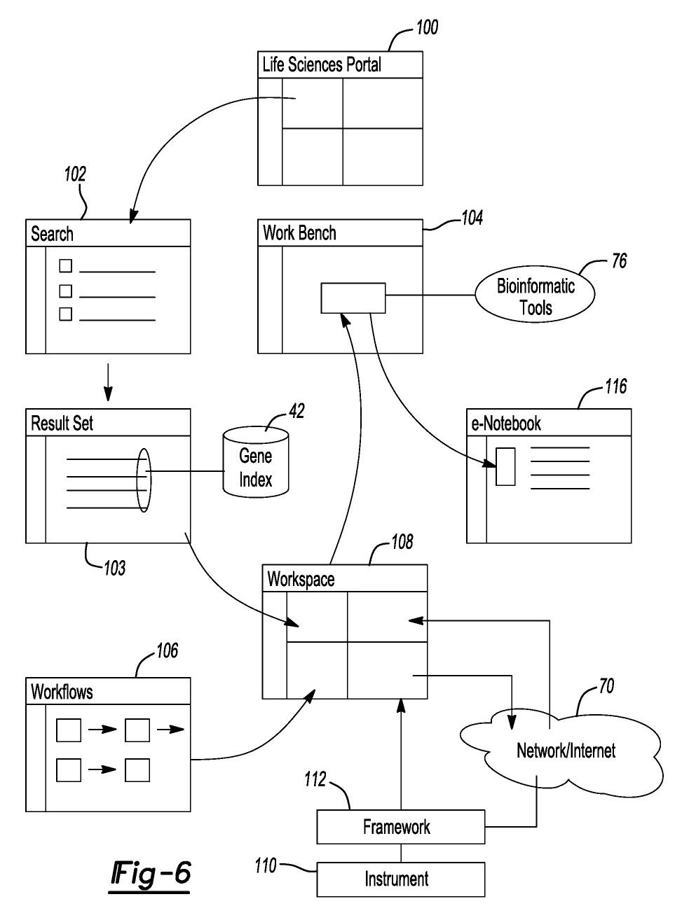

It is anticipated that many practical implementations of the information system 20 will consist of a collection of computer and information systems that are distributed across a network such as the Internet. In this regard, the virtual community 22 may be implemented using one or more servers associated with a service provider, such as bioinformatics service provider 28. As will be illustrated by example in connection with FIGS. 5 and 6, the virtual community 22 may be accessed through a suitable web page interface. Of course, other types of interfaces are also possible without departing from the spirit of the invention.

The illustrated bioinformatics service provider 28 may, itself, have a collection of life sciences information 30 that users of virtual community 22 may have access to. In one presently preferred embodiment the life sciences information 30 may include information at various levels, e.g., genomics, pharmacogenomics, proteomics, cellular biology and cheminformatics information. The information may be extracted from a variety of data sources and in a variety of data formats. Such formats include, but are not limited to: the FASTA format, the GenBank/EMBL/DDBJ format, the SWISS-PROT format, the Pfam format and the PROSITE format.

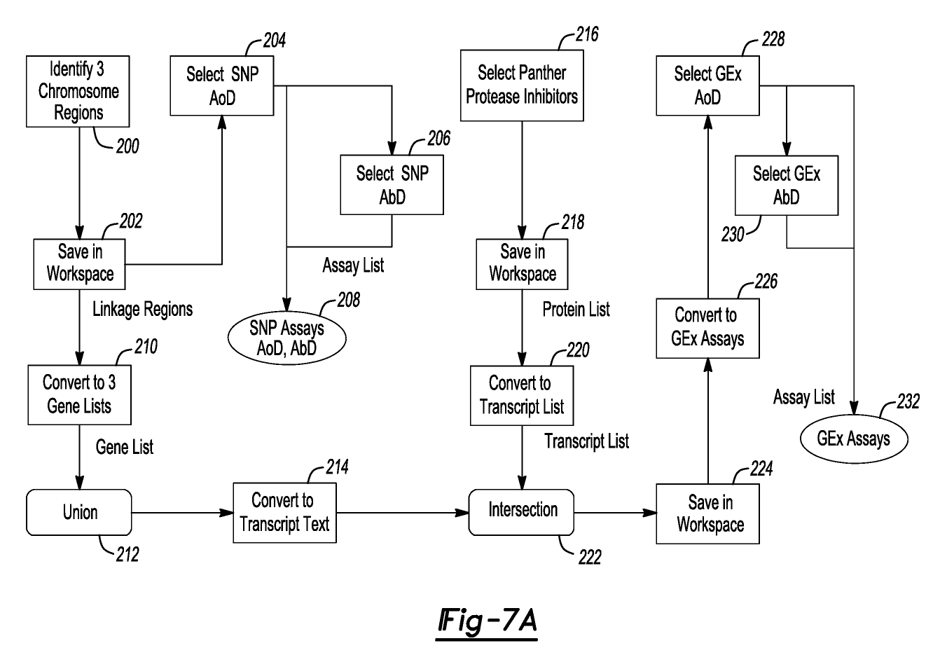

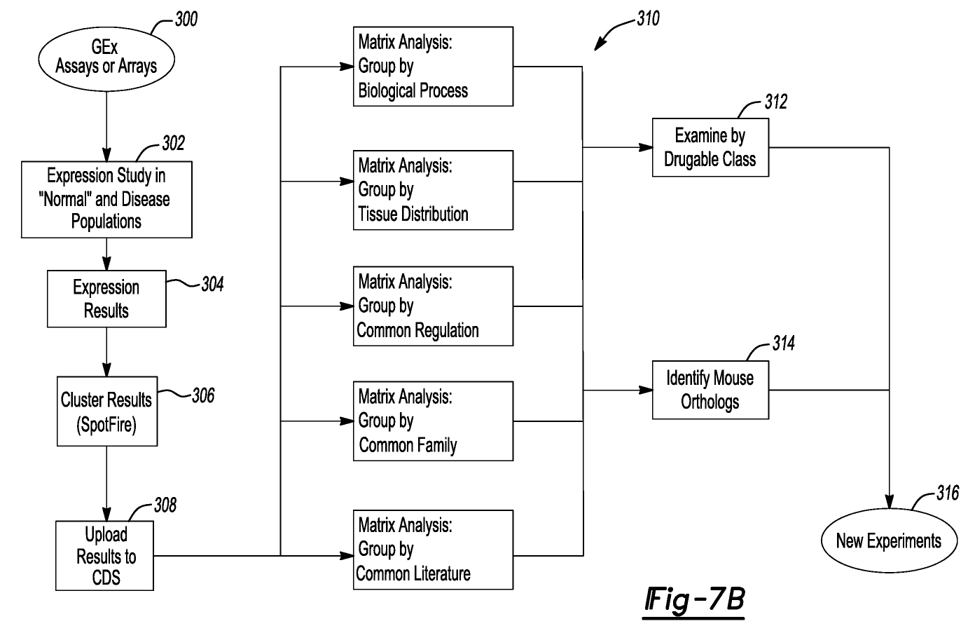

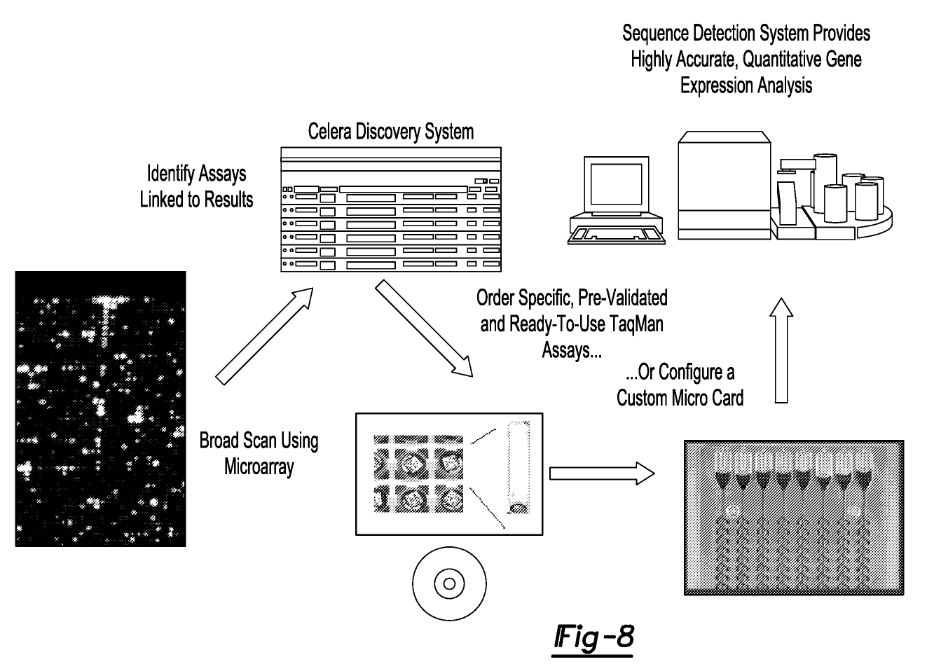

Bioinformatics service provider 28 may also have a collection of predefined workflow patterns 32 that are made accessible to users of virtual community 22 for use in conducting biological and life sciences research. Examples of such workflow patterns will be presented in connection with FIGS. 7 and 8 below. In addition, the bioinformatics service provider 28 may also provide access to research appliances, illustrated diagrammatically at 34. For example, the bioinformatics service provider may provide access to research appliances, such as gene sequencers, DNA microarray readers, and the like, to users of virtual community 22. In accordance with one aspect of the technology, a life sciences framework may be used to implement a common accessing methodology for such research appliances. Such a framework is desirable because it allows users within the virtual community to obtain information from the research appliances, without concern for constructing a compatible local hardware/software environment. The framework defines a suitable hardware/software interface structure and application program interface (API) to allow a diverse collection of research appliances from different manufactures to communicate with one another and with the users of virtual community 22.

The virtual community 22 is preferably configured so that its users can also access resources that are not necessarily associated with bioinformatics service provider 28. Thus, users of virtual community 22 can access life sciences information 36, workflow patterns 38 and research appliances 40 that are made available on the network by third parties or by other members of the virtual community 22.

As will often be the case, the biological or life sciences researcher will have a particular technical discipline or technical field of endeavor that defines much about that researcher's experiments conducted in virtual laboratory 24. However, the biological and life sciences represent a vast body of knowledge that spans numerous scientific fields of endeavor. The virtual community 22 is designed with this in mind. Thus, as illustrated at 22a, the virtual community 22 preferably comprises an N-dimensional space that may be diagrammatically depicted as layers each corresponding to a different biological or life sciences discipline. At 22a, the following disciplines are illustrated: genomics, pharmacogenomics, proteomics, cellular biology and cheminformatics. The virtual community 22 is configured using an information indexing system that allows a researcher working primarily in one field of endeavor (one layer) to “tunnel” up or down to access resources or information that are defined primarily for other disciplines (other layers). Thus, a genomics researcher can use the virtual community 22 to acquire proteomics information that may be useful in an experiment that research is conducting in the genomics field.

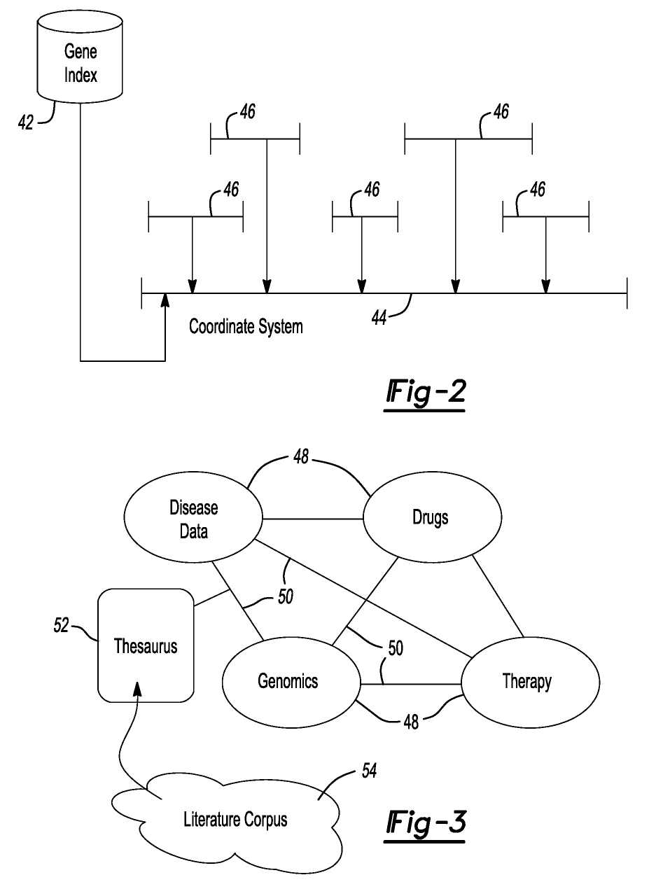

FIGS. 2 and 3 show how the virtual community is indexed to allow researchers to tunnel through to different layers of information. Referring first to FIG. 2, the preferred embodiment employs a gene indexing system illustrated diagrammatically at 42. The unifying principle employed by the gene indexing system 42 is establishment of a unifying coordinate system 44 onto which various discovered genes 46 are mapped. The presently preferred embodiment uses the applicable genome, itself, as the coordinate system 44. Thus, the human genome is used as the preferred coordinate system 44 for human research. The gene indexing system may thus be viewed as a genomic catalog that performs data merging or data integration among a variety of different genomic data sources. Thanks to the unifying coordinate system 44, the gene indexing system 42 is capable of merging or integrating genomic data from a variety of different sources (i.e., from other databases), including the Celera database, the Genbank database, the Swiss-PROT database, and the like. It will be appreciated that these databases were developed by different research groups, with different goals and objectives, and thus the information in one database does not necessarily map to information in another database, without the coordinate system of the gene index 42.

The gene indexing system of a presently preferred embodiment builds upon the coordinate system 44 to include relational links among diverse collections of information that correspond to the information layers illustrated at 22a in FIG. 1. A presently preferred data structure for capturing these associations has been depicted in FIG. 3. In FIG. 3, various different information domains have been illustrated by reference numerals 48. The relationships among these domains have been illustrated by reference numerals 50. Associated with each relationship 50 is a thesaurus 52 that defines a relationship between an information component in one information domain and an information component in another information domain. In this regard, an information component may comprise any information expressed by alphanumeric text, including, words, phrases, gene sequences, and the like. In the presently preferred embodiment, thesaurus 52 is developed using the corpus of published literature 54. The individual entries defined by thesaurus 52 can be developed using computer text-based searching algorithms, with the results thereof being refined by human curation.

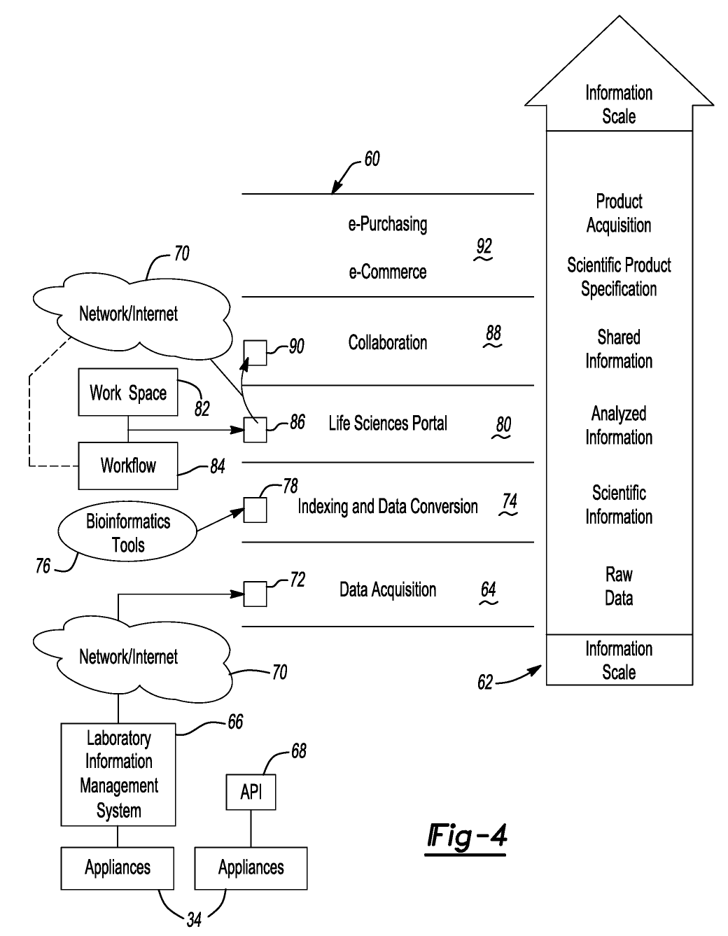

The information system employs a layered information architecture illustrated at 60 in FIG. 4. The architecture organizes information along an information scale that corresponds to successively more refined information content. This information scale is illustrated at 62.

According to the presently preferred information architecture, raw data is acquired at the data acquisition layer 64. As shown by the adjacent information scale, the acquired data is typically raw data of the type produced by research appliances 34. In the illustrated example, two such appliances 34 are shown. One is connected through a laboratory information management system 66 and the other through a suitably configured application program interface (API) 68. The appliances 34 communicate the raw data over a suitable network such as network/Internet 70, thereby making the raw data from appliances 34 accessible to the information system as raw data element 72. Note that the raw data element 72 is initially acquired by the data acquisition layer 64. Thereafter, data element 72 is passed or made available to the indexing and data conversion layer 74 where one or more bioinformatics tools 76 are applied to convert the raw data element 72 into scientific information data element 78.

The scientific information data element represents a higher form of information on information scale 62. It is within the indexing and data conversion layer 74 that the gene indexing system 42 (FIGS. 2 and 3) is utilized. Thus the scientific information data element 78 may be linked, using the gene indexing system 42, to other scientific information maintained by the virtual community.

After processing at the indexing and data conversion layer 74, data element 78 is passed or made available to the life sciences portal layer 80. It is within this layer that much of the analytical work is performed by the researcher. The researcher uses a workspace 82 defined within the virtual laboratory 24 (see FIG. 1). If desired, the researcher may utilize a workflow template 84 that is downloaded or selected by the researcher as a component within the virtual laboratory 24. This workflow template may be acquired from one of the workflow patterns available from the network/Internet 70 (see workflow patterns 32 and 38 in FIG. 1). Depending on the steps performed by the researcher, which may be, in part, dictated by workflow 84, the scientific information data element 78 is converted into an analyzed information data element 86. The analysis performed within the life sciences portal layer 80 may also include the application of additional bioinformatics tools 76.

Once the researcher has completed his or her analysis, the analyzed information data element 86 may be passed to the collaboration layer 88 where that element may be made available to others as a shared information data element 90. The shared data element 90 may be made available to others by placing it in a public location or shared workgroup location within the virtual community 22 (FIG. 1). In a presently preferred embodiment the data elements 72, 78, 86 and 90 can be stored within the virtual community using the virtual data store 26 (FIG. 1). This virtual data store can be configured in different ways to affect the desired data security model. In one embodiment the virtual data store is implemented on servers maintained by a service provider such as the bioinformatics service provider 28 (FIG. 1). Preferably the data are maintained in an encrypted form with suitable authentication protocols in place to protect the information from being distributed to others without the information creator's authorization. In another embodiment, the virtual data store 26 is implemented as a collection of pointers for uniform resource locator (URL) identifiers that designate a storage location on the information creator's computer system. In this latter embodiment, the data developed while using the virtual laboratory 24 are stored on the user's computer systems and are hence not available to others on the public Internet unless the user's system administrator so permits.

Referring again to FIG. 4, the information architecture 60 includes yet another layer 92 upon which e-commerce and e-purchasing applications may be built. In a presently preferred embodiment the user, following the steps outlined in workflow 84, may, from time to time, need to specify scientific products that may be useful or necessary in conducting research. Such product specification is typically associated with the specific workflow template being utilized, and also based on the actual data element or elements being worked with. Thus the top layer 92 includes the ability to make specific product selections, and these selections are then input into a product acquisition electronic purchasing system. Preferably, the purchasing system is designed to conform to the purchasing requirements of the researcher's institution or company. This may entail, for example, integrating with or passing data to a company-wide or institution-wide purchasing system.

The information system illustrated in FIGS. 1-4 may be implemented in a variety of different ways. One presently preferred embodiment employs web technology to present the virtual community through a life sciences portal. FIG. 5 illustrates an exemplary embodiment of a life sciences portal. The portal is accessed through a main page or home page 100, to which a plurality of additional pages are linked, namely a search page 102, a workbench page 104, a workflow page 106 and a workspace page 108. The workspace page provides data connectivity with research appliances and instruments, such as instrument 110 by utilizing the life sciences framework 112. Additionally, the workspace 108 can be made selectively visible to others via the network or the Internet and may also be used to import information from other systems. This functionality is illustrated diagrammatically at 114.

Each of the aforementioned pages or screens provides a different type of functionality, which will now be explained through the use case example illustrated in FIG. 6. The researcher enters the information system through life sciences portal 100. In this case, the researcher first accesses the search page 102 where he or she conducts an information search across the available life sciences information (such as information 30 and 36 in FIG. 1). The results of the search are then displayed as a result set in screen 103. Note that the results may be associated or linked to one another through the gene indexing system 42. Thus the researcher, performing genomics research, may uncover associated information identified with the proteomics domain, or the cellular biology domain, for example. (See FIG. 1 at 22a.)

The researcher then selects all or a portion of the result set and places it into the workspace page 108. To assist the researcher in a systematic analysis, a suitable workflow template may be loaded into workspace 108 by accessing the workflow's page 106. In addition, the researcher may elect to couple his or her in silico research (contained on workspace page 108) to a research appliance or instrument 110. In this regard framework 112 provides the necessary control and data connectivity to allow the user to control and obtain raw data from instrument 110 without the need to directly invoke the instrument control functions in the native instrument's control language. Rather, framework 112 provides a universal structured control language by which instrument 110 may be controlled and the results transmitted to the storage location specified by the researcher on the workspace page 108. The actual data storage may be assigned to a storage location associated with the virtual data store 26 (FIG. 1) or a different storage location specified by the researcher. In some applications the framework 112 may communicate data directly to the workspace page 108. In other applications the framework 112 communicates through the network or Internet 70.

Workspace page 108 can be used to perform many of the information processing tasks associated with the layered information architecture shown in FIG. 4. In this regard, obtaining information through the search page 102 or from a research appliance or instrument 110 represents part of the raw data acquisition layer 64. By virtue of the gene indexing system 42, this raw data is converted into useful scientific information that the researcher then analyzes and optionally shares. The sharing of information corresponds to the collaboration layer 88 of FIG. 4. It is effected in the embodiment of FIG. 6 by making selected portions of the workspace page 108 accessible to other users over the Internet 70.

In some instances a given workflow template will specify that certain bioinformatics tools 76 should be utilized upon the data set being analyzed within the workspace page 108. Such analyses can be performed within workspace page 108, however, a presently preferred embodiment allocates the more computationally intensive bioinformatics tasks to a separate page designated as the workbench page 104. Results of bioinformatics processing effected on workbench page 104 can be sent back to the workspace page 108, or optionally, to an electronic notebook page 116. The electronic notebook page provides the researcher with a convenient place to store personal notes about his or her research that are not necessarily intended for sharing within the workspace page 108.

Much of the power of the information system lies in its ability to integrate information from diverse sources, across multiple scientific disciplines, and to coordinate experimental research through workflows. To further illustrate these concepts, two exemplary workflows will now be described in connection with FIGS. 7 and 8. The workflow of FIG. 7 represents an experimental design workflow that might be used by a life sciences researcher in coordinating genetic experiments. The workflow of FIG. 8 is a data analysis workflow, corresponding to one that might be performed using bioinformatics tools to analyze a data set. Both of these illustrated workflows might be provided as templates for uploading into the workspace page 108 (FIG. 6) to guide research. To follow the workflow of FIG. 7, being in the upper left hand corner at 200. The researcher identifies three chromosome regions at 200 and these are saved, at step 202, in the workspace. The saved chromosome regions may then be used at step 204 and 206 to select SNP AoD and SNP AbD. These selected SNPs comprise an assay list that is stored at 208.

Meanwhile, the chromosome regions saved in workspace 202 represent linkage regions that may be converted at step 210 to three gene lists. A data union operation is performed on the gene lists at 212 and the result is converted at 214 to a transcript text.

Meanwhile, at step 216 the researcher selects Panther protease inhibitors program which can be acquired through the search page 102 (FIG. 5) and these are saved in the workspace at step 218. The saved data from step 218 comprises a protein list that is then converted at step 220 into a transcript list. Now the transcript list produced at step 220 and the transcript text produced at 214 are combined by a database intersection operation 222 and the result is saved at 224 in the workspace.

The saved transcript list is then converted at step 226 into GEx assays and the desired assays (GEx AoD, and GEx AbD) are selected at steps 228 and 230, with the resulting assay list being stored at 232 to comprise the GEx assays list.

Once the assays list is stored, it can be used to access an e-commerce and e-purchasing system to obtain the physical assay kit and associated supplies for conducting wet laboratory research based on the information developed.

In order to accomplish the workflow outlined in FIG. 7, the information system performs a variety of functions. These functions are outlined in Table I below.

TABLE I
Functions Required

Portal Workspace Page
Store objects:
regions
genes
transcripts
proteins
SNP assays
GEx assays
Convert objects:
regions to SNPs
SNP to SNP assay
region to gene list
gene to transcript
protein to transcript
transcript to GEx assay
Set operations:
union
intersection
Portal Search Page
Query operations:
chromosomal regions
protein families
Selection operations:
SNP AoD
SNP AbD
SNP AoD
GEx AoD
GEx AbD
Commerce operations:
order assays

The data analysis workflow example of FIG. 8 begins in the upper left hand corner at 300. At step 300 a set of GEx assays or arrays is provided. An expression study is then conducted at 302 corresponding to both normal and diseased populations. The expression results are then obtained and stored at 304 and the results are clustered at 306 using a suitable clustering algorithm such as the SpotFire. The results are then uploaded at step 308 to an information system such as the Celera Discovery System (CDS) facility, making the results available for a collection of different matrix analysis operations illustrated collectively at 310. The results of the matrix analysis can lead to additional steps such as an examination step 312 where the results are explored by drugable class, and at step 314 where other orthologs are identified (e.g., mouse orthologs). The results of processing steps 312 and 314 then suggest new experiments, as illustrated at 316.

The functions required to perform the data analysis workflow of FIG. 8 are set forth in Table II below.

TABLE II
Functions Required

Portal Workspace Page
Store objects:
genes
transcripts
proteins
Set operations:
union
intersection
Functional operations:
upload genes
launch application
CDS operations
Matrix analysis:
biological process
tissue distribution
chromosomal location
regulation
Classification:
drugable class
orthologs

Another workflow illustrated in FIG. 8 demonstrates how researchers can be guided through a research process. For example, the workflow includes starting with a broad scan using a microarray, and then identifying assays linked to results of the broad scan using the Celera Discovery System. Next, the researcher may select and perform one or more of a plurality of assays, including ordering specific, pre-validated and ready-to-use Taqman Assays, and including configuring a custom MicroCard. Finally, the workflow instructs the researcher to analyze the results with a sequence detection system to provide highly accurate, quantitative, gene expression analysis. Other workflows are also available to assist researchers in identifying SNPs that are useful to their research, and to perform steps before and after the SNP identification to achieve the proper results.

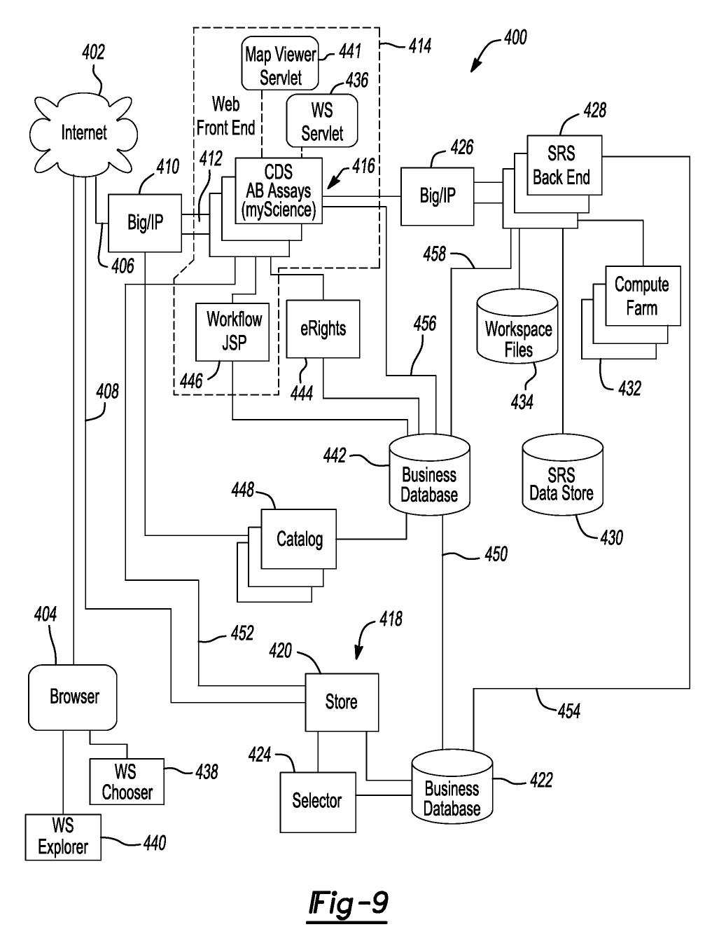

Referring to FIG. 9, an exemplary hardware implementation of the information system has been illustrated. It is to be understood that the embodiment illustrated is merely intended as one example of a possible implementation. Those skilled in the art will appreciate that other configurations are also possible.

In the illustrated hardware implementation, users interact with the information system 400 by access over the Internet 402 using a suitable browser 404. The information system 400 is coupled to the Internet as at 406. Although a single Internet connection may be utilized, the illustrated embodiment illustrates how a second Internet connection as at 408 can be employed to connect different parts of the information system to the Internet. As illustrated, connection 406 couples a portion of the server subsystems through a distribution server 410, also designated as Big/IP 410. Big/IP system 410, in turn, supplies multiple TCP/IP connections as at 412 to the web front end system 414. Web front end system 414 comprises a plurality of servers that may be configured to provide different website functionality. In FIG. 9, a plurality of servers 416 have been illustrated. For illustration purposes, servers 416 have been labeled CDS, AB Assays and myScience. These designations illustrate possible web server systems, such as the Celera Discovery System (CDS), the AB Assays System and the myScience System that are all implementations of websites operated by the Assignee of the present patent application.

Internet connection 408 couples to an e-commerce system 418, that includes an e-commerce store server 420, a business database 422 and a selector server 424 that functions to integrate the store server with the business database.

The lab front end 414 is coupled through a second Big/IP system 426 to a sequence retrieval system 428. The sequence retrieval system (SRS) includes a data store 430 containing gene sequence data. The SRS system 428 is coupled to a collection of servers identified as the compute farm 432. These servers perform various bioinformatics processes upon the sequence data within data store 430. For example, the compute farm could perform a BLAST search upon the sequence data.

Associated with the SRS back end system 428 is a workspace file structure 434 into which the user workspace information is stored. In the illustrated embodiment, the workspace file structure 434 allows workspace information to be conveniently stored for later retrieval and use by the user through browser 404. In this regard, the web front end 414 includes a workspace servlet 436 that provides workspace manipulation functionality at the browser 404. In the illustrated embodiment, the servlet 436 provides workspace chooser functionality within browser 404, as illustrated at 438. Servlet 436 also provides workstation explorer functionality at 440. The chooser functionality 438 allows a user to identify locations within the workspace file structure 434 for saving information. Conversely, the explorer functionality 440 gives the user access to the workspace files 434 for information retrieval and subsequent manipulation operations such as moving or renaming information.

Other functionality may also be provided using servlet technology. Thus, as illustrated, the map viewer functionality may be provided using servlet 441. The map viewer will be illustrated in greater detail below.

The information system 400 further includes a business database 442 that is used to store user information and session information as well as system utilization information. Access to the information system 400 is mediated by an access control module identified as eRights server 444. The eRights server is coupled to business database 442 and also to the web front end 414. In an exemplary embodiment, the system provides different levels of user access. In a first level a user is entitled to only view certain information available through the various websites available to the web front end 414. At a next higher level a user is authenticated and given access to additional functionality, which may include access to workspace files within workspace file structure 434 and access to other features of the system as previously described. At a third and yet higher level the user is also given access to certain premium data files, such as data files associated with the Celera Discovery System (CDS). The eRights server 444 is utilized to ascertain the user's identity, authenticate the user and then grant the user access to whatever level of use the user is entitled to enjoy.

As previously described, the information system 400 provides a useful set of workflow tools or protocols that allow the researcher to organize his or her research and to integrate that research with the work of others. This workflow or protocol functionality is provided by a workflow JSP (Java Server Page) server 446 that is coupled to the web front end and also to the business database 442. Workflows or protocols are stored by the workflow JSP server 446 and may be served to selected web pages or frames within web pages on the user's browser 404. Additionally, workflows may be downloaded to a user's biotechnology instrument, personal computer, or networked instrument system. As previously described, these workflows identify predetermined steps that a user of the system may wish to follow when conducting research. At each step, the user is presented with convenient information and/or access to the e-commerce systems to purchase materials needed for conducting further research.

The e-commerce system illustrated in FIG. 9 at 418 works in conjunction with a catalog data store 448 in which the product offerings of affiliated companies are cataloged for identification and purchase. This catalog datastore may be detachable and distributable, such that the catalog may be incorporated into other web sites and/or downloaded by a user onto an instrument, personal computer, and/or networked instrument system. Although the catalog data store 448 has been illustrated in association with business database 442, the same catalog information is available to the store 418 through business database 422. As illustrated, business database 422 is coupled to business database 442 through a suitable data connection such as a Virtual Private Network (VPN) connection 450. Data may be synchronized between these two databases in batch mode, for example. In this way, product catalog information can be propagated throughout the system, as well as user data. Because user data has certain real-time aspects, the illustrated system of FIG. 9 includes additional VPN connections at 452 and 454 to couple business database 422 with the respective web front end 414 and SRS back end 428. Thus real-time synchronization is provided, as required, between the web front end system 414, the back end system 428 and the e-commerce system 418.

As previously discussed, the system is capable of providing collaboration among users to promote virtual communities and to foster more advanced research. Sharing of information is possible through the workspace files 434. This may be implemented using the eRights server 444. The eRights server can give any designated user access to another designated user's workspace files. In this way, those two users can collaborate with one another. The eRights server 444 can also give access to selected users to the workflow JSP server, to allow authenticated users to upload and thereby share workflows with one another. The uploaded workflows would be stored in business database 442, for example.

In the illustrated implementation, there are various protocols by which data may flow. The SRS back end 428 may be configured to provide HTML data that is then proxied through the web front end 414 for display on one or more of the web server sites within the web front end. Alternatively, the SRS back end and web front end may communicate with each other using XML data. In this use, the web front end 414 treats the SRS back end 428 as a data store from which it retrieves information for display on one or more of its websites. In addition, the web front end 414 and the SRS back end 428 are both configured to communicate through respective connections 456 and 458 with the business database 442. Such communication may be by direct SQL query, for example.

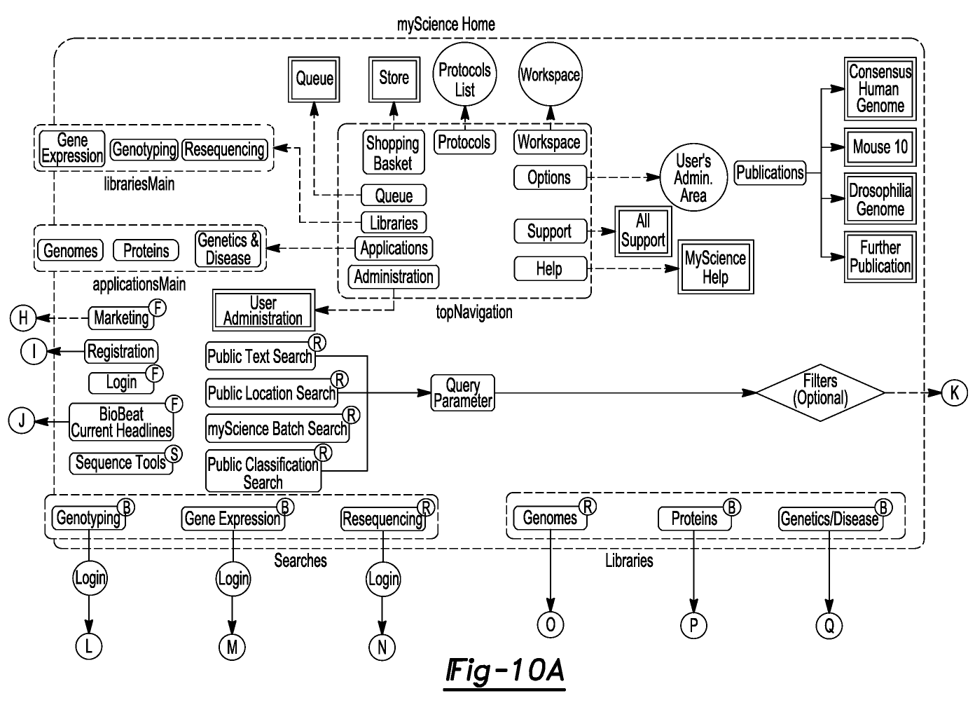

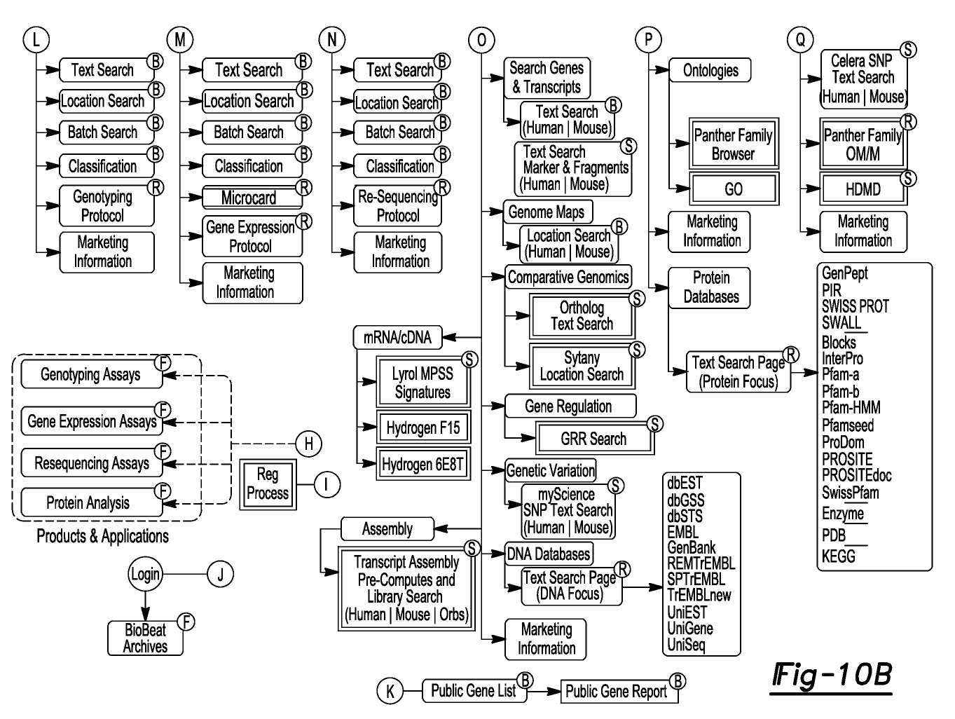

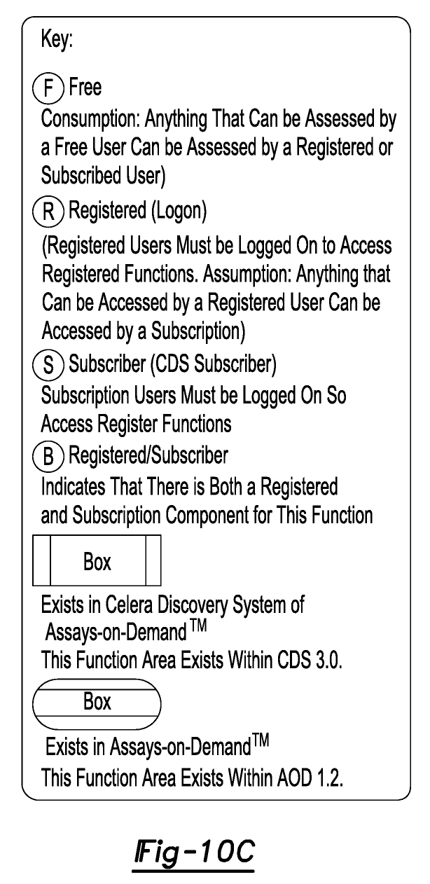

Having thus described an exemplary hardware embodiment of the information system, an exemplary web portal implementation will now be described. In this regard, it will be appreciated that any web implementation involves design decisions regarding how the site will appear and how the user will navigate the site. Thus, the illustrated embodiment shown in FIGS. 10-19 are intended only to illustrate the principles involved and should not be construed as a limitation upon the scope of the invention as set forth in the appended claims.

An exemplary workflow map is shown in FIGS. 10A and 10B. As has been previously suggested, access to a sophisticated information system may be managed by giving different classes of users different access rights. The eRights server 444 of FIG. 9 may be used to mediate this functionality. In FIGS. 10A and 10B, different navigational endpoints have been designated by the letters F, R, S and B to depict different exemplary classes of users. For example, the F class corresponds to free users who can access only a minimal set of pages and features within the system. The R designation corresponds to registered users who have logged on and thereby authenticated themselves to the system. Such users can view some content and enjoy some features that the free user cannot. The S designation corresponds to users who are subscribers of the system and are thus given access to premium content, such as access to the Celera Discovery System (CDS). Finally, the B designation corresponds to content that both registered users and subscribers may access.

The access control system thus implemented allows different levels of content to be provided to different levels of users. For example, proprietary genome data may be provided solely to subscribers based on the need of the subscribers for privacy in their research, and based on contractual obligations relating to the proprietary nature of the data and its use. Also, publicly available genome data may be provided to all users as this data could be accessed alternatively through other sources. Further, the registration process allows users to be accurately identified, so that related users may share a common workspace, while privacy is still maintained. Thus, the system can provide users who are reluctant to have their research patterns tracked by others monitoring Internet traffic the capability to perform research in a secure environment. Simultaneously, the system can service other users accessing publicly available data.

As illustrated in FIG. 10A, the homepage, designated as myScience may be explored by navigating from the top navigation choices through to the various products and applications sites, libraries sites, search sites, and the like of FIG. 10B.

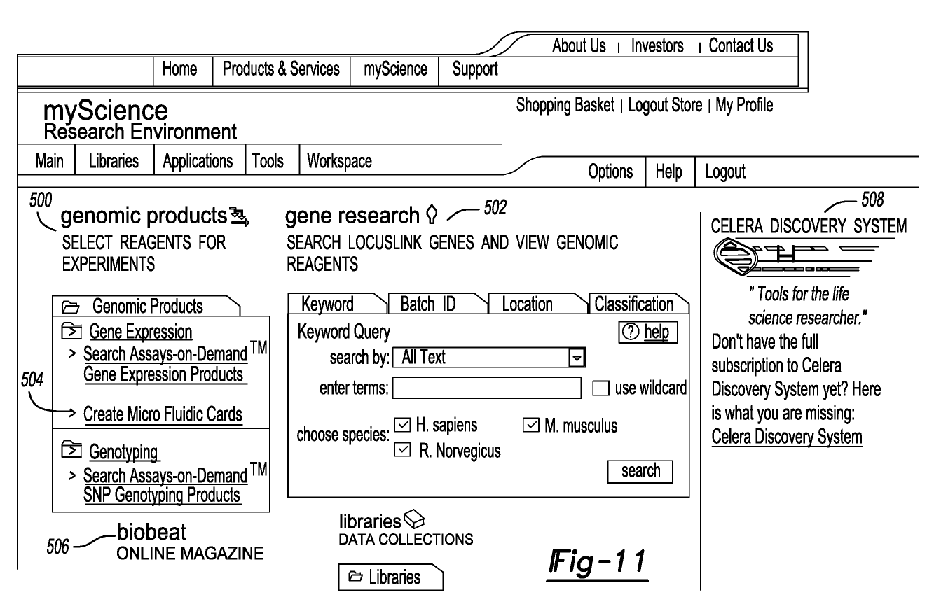

FIG. 11 shows an example of a homepage corresponding generally to the myScience homepage depicted in FIGS. 10A and 10B. The exemplary myScience website provides an example of an online life science research environment and virtual community, with a focus on design and analysis of biological experiments. The user can use the website to conduct research, such as to search for genomic products. This would be done by accessing the portion of the site depicted at 500. Alternatively, or additionally, the user could search for specific genes, such as searching for specific human, mouse or rat genes by keyword, ID, genomic location or protein classification. Such a search would be input in the section of the website at 502, for example. This search would also be capable of returning associated genomic products applicable to that gene search. Still further, the user could access a link within the site to perform other operations, such as to create a custom configured assay. An example of such capability is shown at 504, where the user can create a micro fluidic card for high throughput custom assay configuration.

In addition to the searching capability, the homepage illustrated in FIG. 11 also provides useful information such as a link to life science community news as at 506. In addition, the site can be used to provide information about additional products or services. In this regard, as illustrated at 508, the user can learn more about a premium feature of the site, in this case the Celera Discovery System.

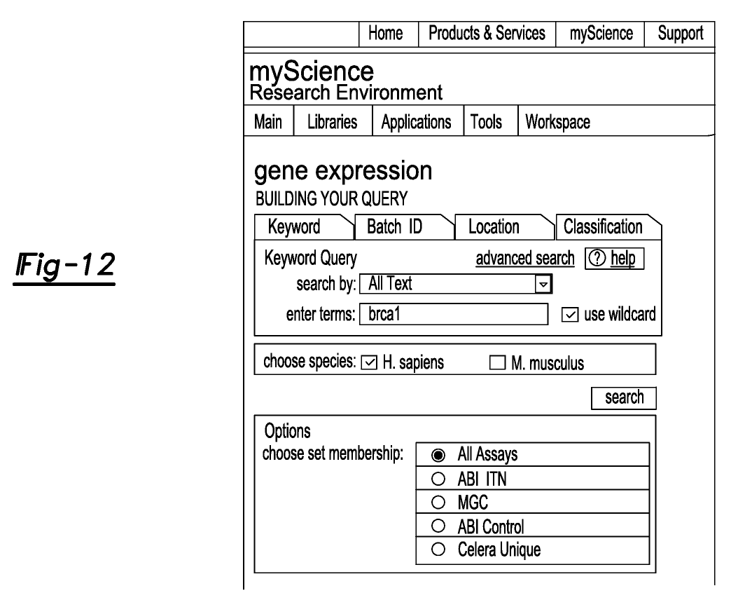

The myScience site provides a research environment that gives users multiple ways to search for genomic information and genomic products. Illustrated in FIG. 12, the user can access the site to enter keywords and then conduct a search for those keywords, based on a particular species. In the illustrated example, the user has entered the term “brca1” and has selected the species to be homosapian.

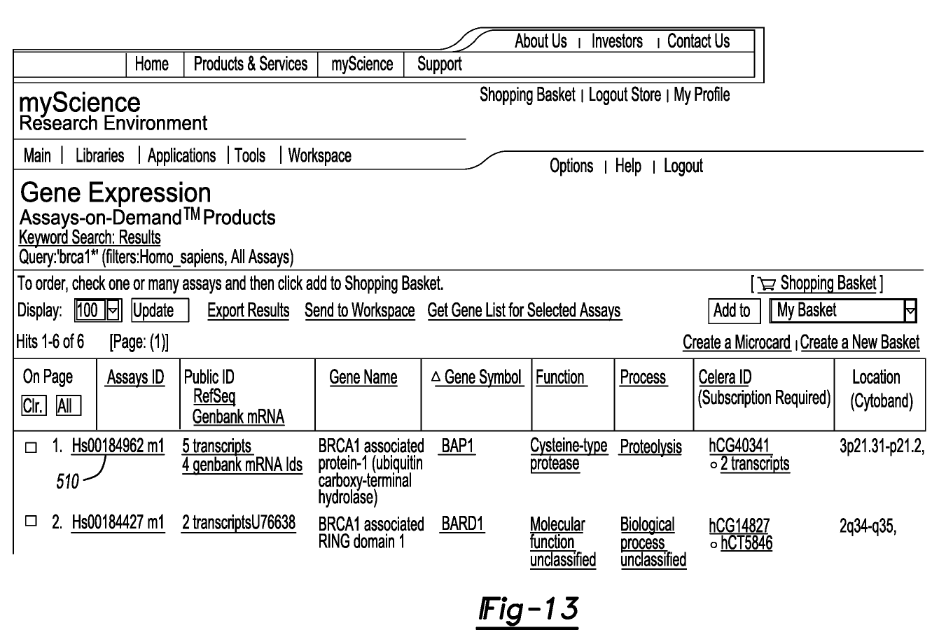

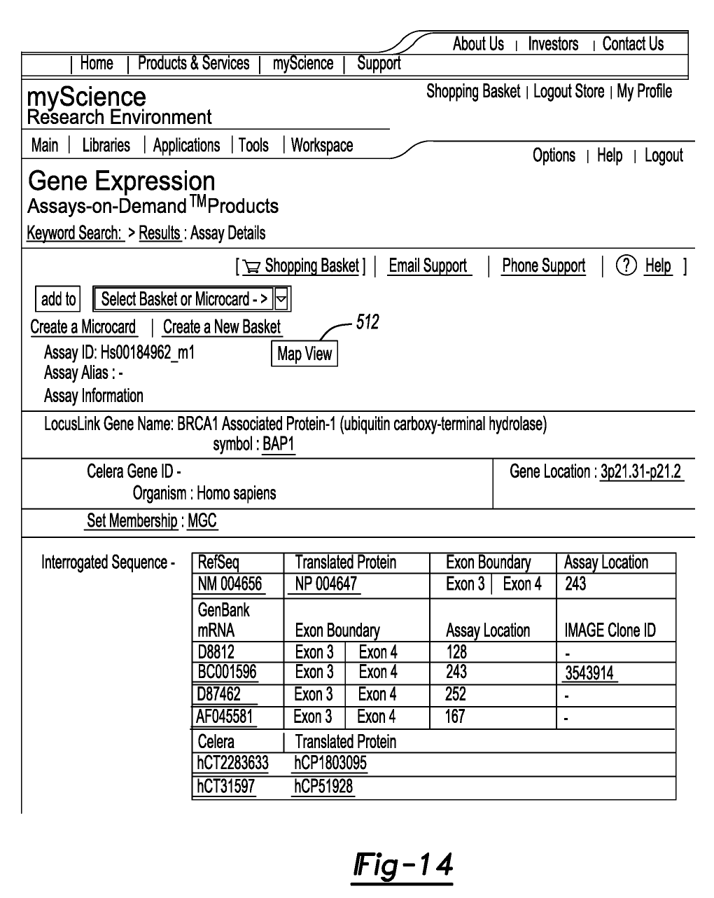

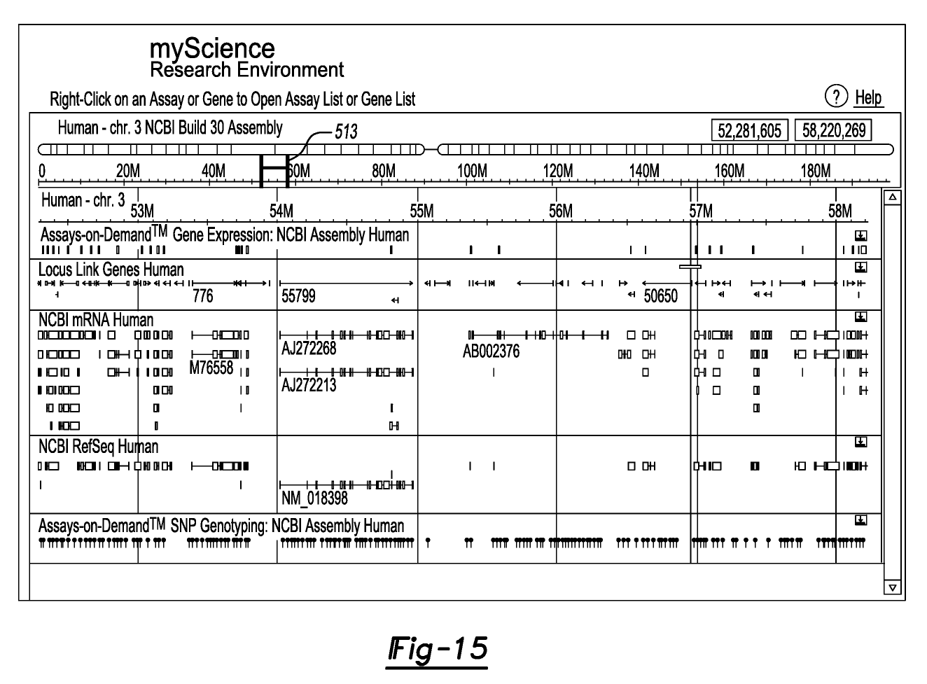

As illustrated in FIG. 13, the system returns a result list screen showing a collection of useful information pertinent to the query that was entered. As illustrated, the result list gives a variety of useful information that is organized to disclose important aspects such as protein function and genomic location for the gene of interest. As shown at 510, the result list includes a hyperlink to an assay detail page from which the user can get additional information. For the illustrated example, FIG. 14 shows the additional information that can be made available. Specifically, the detailed information includes information about the selected assay, such as interrogated sequence, gene location and protein function. The user can easily explore this in more detail by clicking on the map view button 512. Doing so, brings up the map viewer display screen shown in FIG. 15.

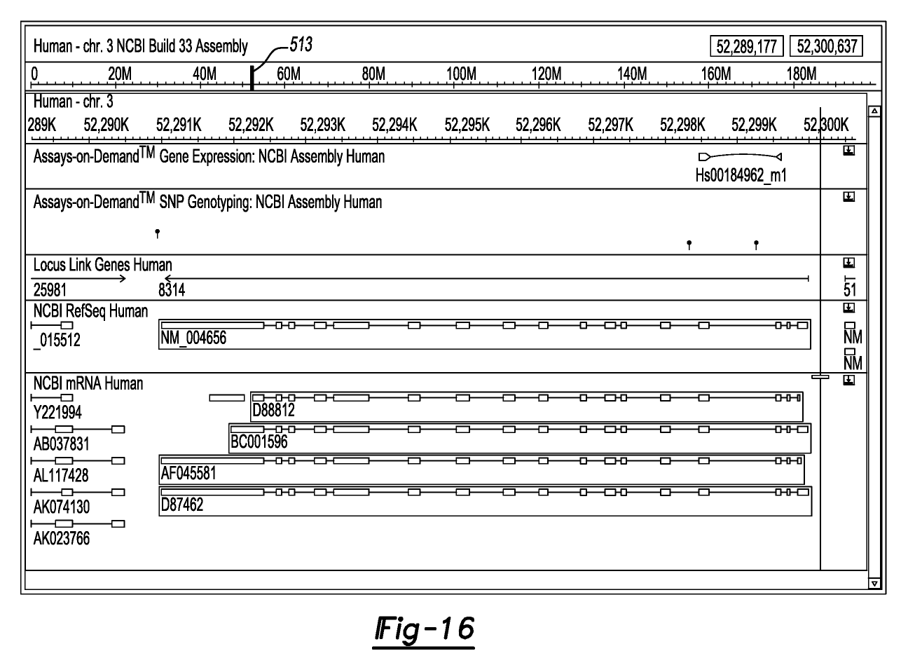

As illustrated, the map viewer correlates visually the selected information at different hierarchical levels. The user can readily expand or contract the view to “zoom in” or “zoom out” as needed by view control 513. In this regard, FIG. 16 shows what the screen might look like if the user zooms in to see more specific detail about the particular assay of interest. A representation of the zoomed portion of the genome is thus displayed below the genome, with known gene introns and exons of the zoomed portion clearly identified. The gene expression assays and SNP assays are further codisplayed in a position relative to the areas of the zoomed portion of the genome to which they relate.

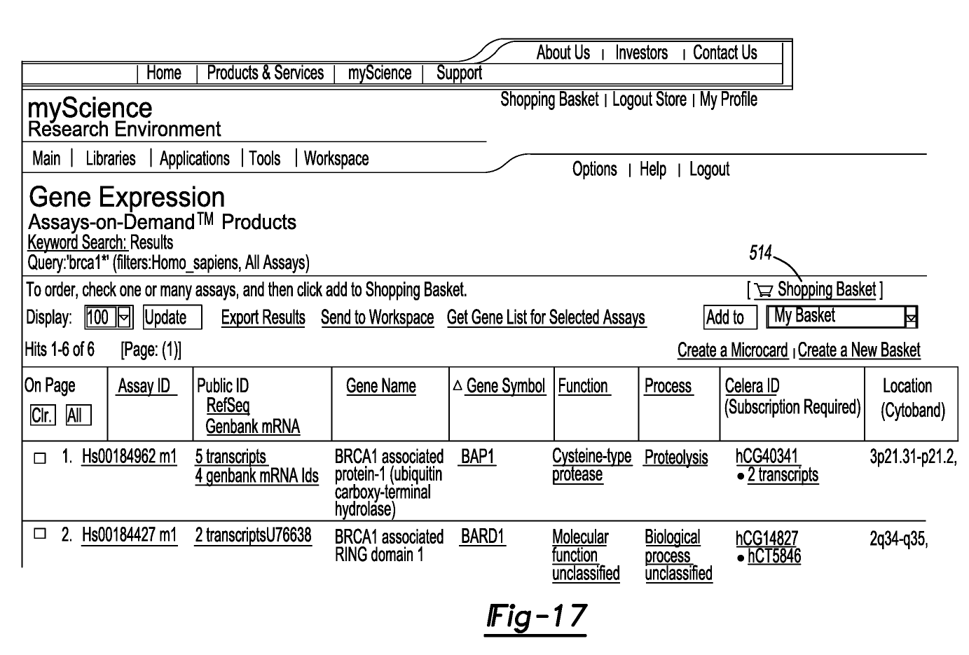

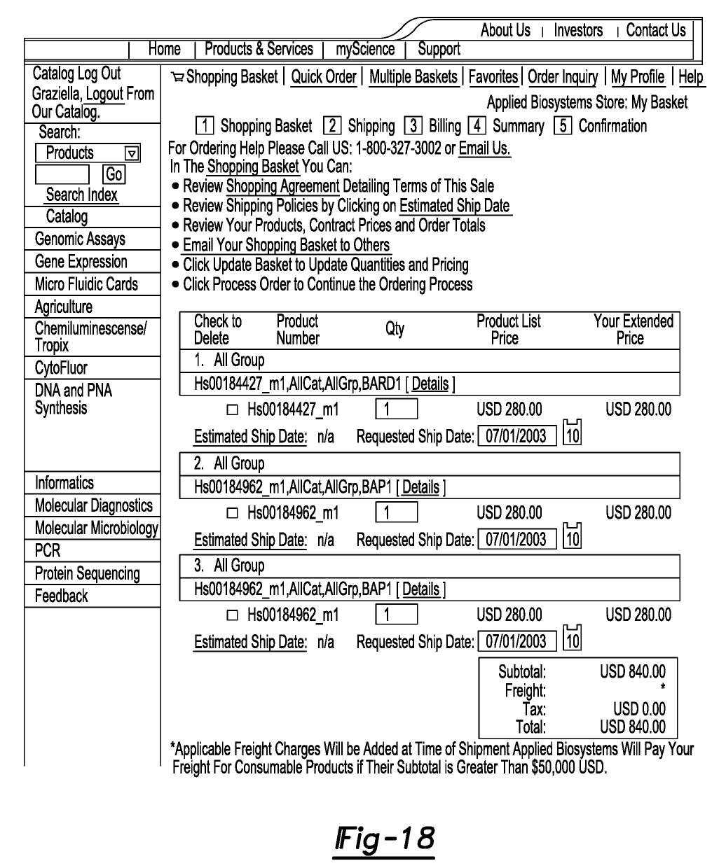

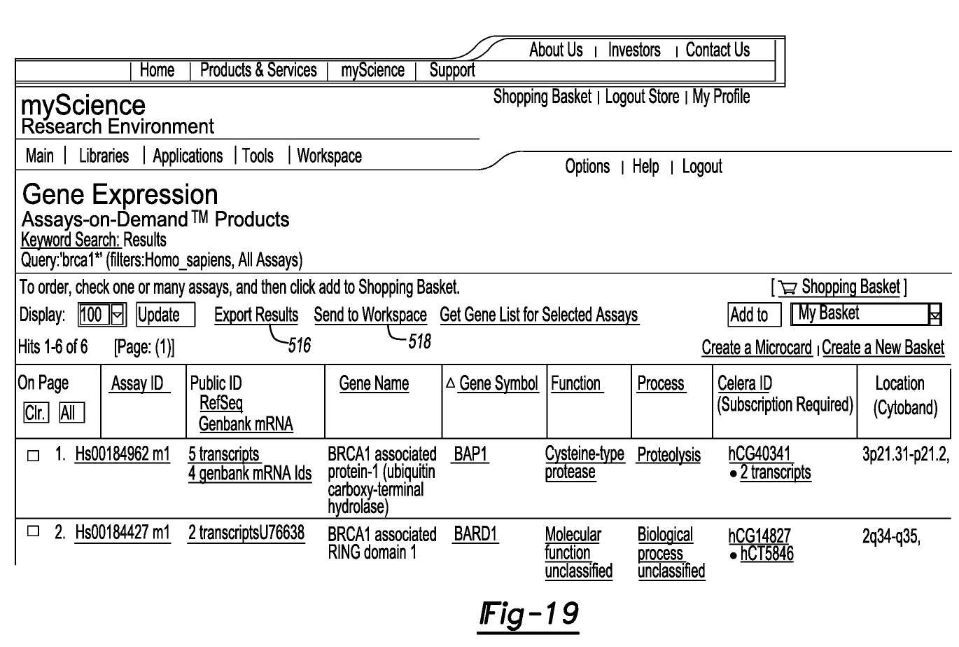

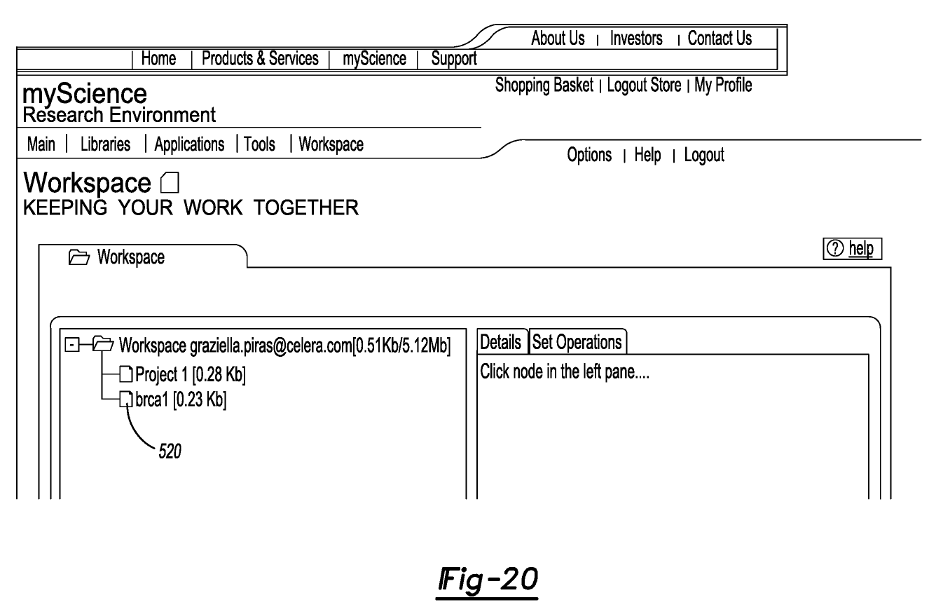

Once a useful assay had been identified, the user can conveniently select it for purchase by clicking on the assay and interacting with the shopping cart basket as depicted at 514 in FIG. 17. Thereafter, as shown in FIG. 18, the user is given an opportunity to review the items in the shopping basket and then to place an order for the selected assay online. The search tools, map viewer and e-commerce systems are integrated as illustrated above, to provide the user with a very convenient way to identify products or services that may be useful in research. However, the integrated website provides even more functionality than this. As illustrated in FIG. 19, the user can manage his or her research results by exporting the research results to the user's personal workspace. This is done by selecting the export results hyperlink 516 to export the results list in tab-delimited format for further analysis. Alternatively, the user can user hyperlink 518 to save the research in the user's personal workspace. Once saved in the personal workspace, the user can conveniently manage the data as illustrated in FIG. 20. FIG. 20 shows at 520 the results of the user's research with respect to the query “brcal1”.

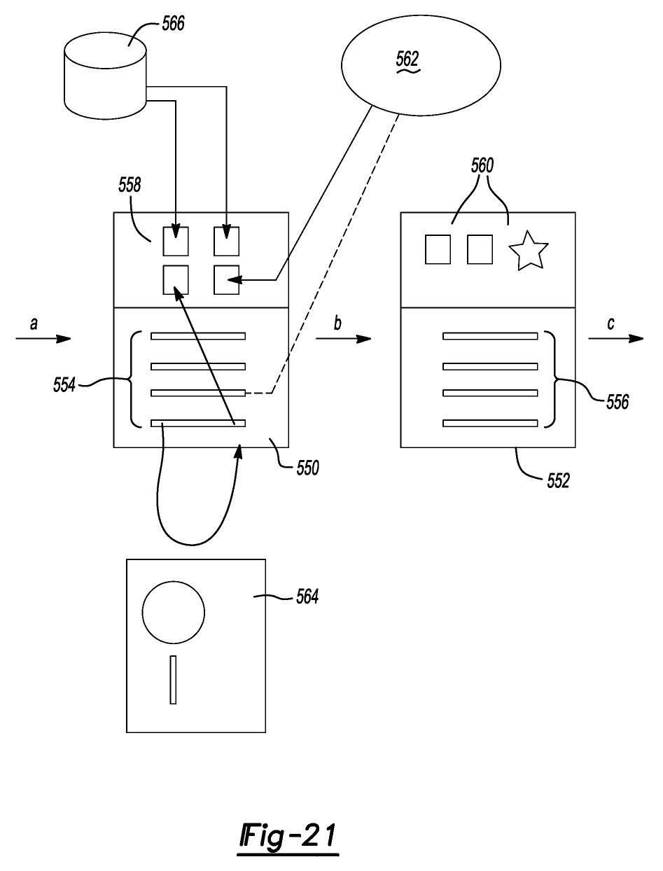

In addition to the functions and features described above, the information system supports a rich environment for creating and sharing workflows to assist the researcher and to promote collaboration. If desired the information system can be implemented to include a workflow framework having tools with which a user can create new workflows and modify existing workflows. Such a workflow framework embodiment is shown in FIG. 21. The workflow may be configured as a linked list of workflow stages. In FIG. 21, two workflow stages 550 and 552 have been illustrated. Each stage may be implemented as a software object or component having a list of steps to be performed or rules to be applied. These steps or rules are illustrated diagrammatically at 554 for stage 550, and at 556 for stage 552. In addition, the object or component representing each workflow stage may have data members for storing data being used within that stage. In FIG. 21, the data members of stage 550 are shown at 558, and the data members of stage 552 are shown at 560. These data members may store actual scientific or operational data or pointers to scientific or operational data.

The stages also include linking variables with which one stage is linked to another, as illustrated by the workflow arrows a, b and c in FIG. 21. These linking variables permit stages to be linked for both forward and backward traversal, as may be required by a particular workflow. Typically, workflows are designed for forward traversal (e.g., stage 550 is made active and its steps performed before stage 552 is made active.

The individual steps or rules within each stage can be used to effect a variety of different operations or data manipulations. The steps may be either passive steps, which merely provide instructional information to the researcher, or active steps, which perform or launch data manipulation steps performed by the researcher's workstation or elsewhere.

In FIG. 21, for example, the third step accesses a remote data store 562, such as a database on the internet, to retrieve data that is stored locally in one of the data members 558. The fourth step accesses an external instrument 564 to receive data that is processed as part of that step and the result stored in another of the data members 558. Additional data are loaded from a data store 566 that may, for example, be a data store of research data maintained by the research institute or company performing the workflow.

According to the workflow framework, the individual workflow stages may be stored as separate objects or components that may be linked together in a variety of different ways, to create new workflows, or to modify existing workflows. In addition, the individual data members and the associated steps or rules can be edited or modified by a user to create new workflows or to modify existing workflows. The framework can be implemented in a variety of different software platforms. If desired, the workflow stages, and the associated objects, components, steps and rules may be expressed using XML. This XML description of a workflow thus defines the workflow in terms of the workflow stages involved. From this description the actual implementation or instantiation of the workflow is constructed and made available to end users via the portal described above.

The description of the invention is merely exemplary in nature and, thus, variations that do not depart from the gist of the invention are intended to be within the scope of the invention. Such variations are not to be regarded as a departure from the spirit and scope of the invention.

1-8. (canceled)

9. A life sciences network portal system, comprising:
at least one networked computer system that defines a portal through which users may access said networked computer system to conduct life sciences research;
a workflow system associated with said portal and operable to allow a user to prescribe and track the performance of a series of steps associated with that user's life sciences research;
a data store of life sciences information accessible through said portal;
a product specifying system accessible through said portal and offering products useful in connection with the performance of said series of steps;
an indexing mechanism associated with said networked computer system that mediates relationships among said workflow system, said data store of life sciences information and said product specifying system.

10-13. (canceled)

14. A life sciences workflow management system, comprising:
at least one networked computer system configured to provide a workflow interface to a user through a portal, said workflow interface operable to allow a user to prescribe and track the performance of a series of steps associated with life sciences research;
a data store associated with said networked computer system into which said user stores and organizes sets of user-specified data for bioinformatics processing;
at least one processor associated with said networked computer system that is configured to perform bioinformatics processing upon said user-specified data;
said workflow interface having a user interaction mechanism whereby said user can manipulate user-specified data stored in said data store and whereby said user can control the performance of said bioinformatics processing.

15. The system of claim 14, further comprising a plurality of predefined workflows.

16. The system of claim 14, further comprising a workflow design interface having:
a software object implementer whereby a user may instantiate and link modules representing stages of life sciences research in an ordered fashion; and
a plurality of selectable interactive methods for importing, exporting, and processing user specified data according to user perceived requirements relating to procedures of life sciences research,
wherein the user may import the methods into the stages in a prescribed order, thereby providing steps for completing a stage of life sciences research.

17-27. (canceled)

28. The system of claim 9, wherein said workflow system includes at least one workflow providing step by step instructions to the user relating to life sciences research procedures.

29. The system of claim 28, wherein said workflow system is operable to suggest new experiments based on the user's prescribed series of steps associated with that user's life sciences research.

30. The system of claim 28, wherein said workflow system is operable to identify workflows that include step by step instructions relating to life sciences research procedures that occur before and after the user's prescribed series of steps associated with that user's life sciences research.

31. The system of claim 28, wherein said workflow system is operable to allow the user to create new workflows, modify existing workflows, or share a workflow with other users.

32. The system of claim 28, wherein said workflow system is operable to perform or launch data manipulation steps based on the user's prescribed series of steps associated with that user's life sciences research.

33. The system of claim 9, wherein said product specifying system includes a consumptible identification mechanism adapted to identify life sciences related consumptibles based on data in said data store accessed by the user.

34. The system of claim 9, wherein said product specifying system includes a consumptible identification mechanism adapted to identify life sciences related consumptibles based on the user's position in a workflow.

35. The system of claim 9, further comprising a purchasing subsystem adapted to communicate life sciences related consumptibles to the user, receive a user selection of a consumptible, and process a purchase of the consumptible by the user based on the user selection; and
wherein said indexing mechanism further mediates relationships among said workflow system, said data store of life sciences information, said product specifying system, and said purchasing subsystem.

36. The system of claim 35, wherein the indexing mechanism is synchronized in real-time with the product specifying system.

37. The system of claim 14, wherein said workflow interface is operable to suggest new experiments based on the user's prescribed series of steps associated with that user's life sciences research.

38. The system of claim 15, wherein said workflow interface is operable to identify workflows that include step by step instructions relating to life sciences research procedures that occur before and after the user's prescribed series of steps associated with that user's life sciences research.

39. The system of claim 16, wherein said workflow design interface further allows the user to export the steps for completing a stage of life sciences research to share with other users.

40. The system of claim 14, wherein said workflow interface is operable to perform or launch data manipulation steps based on the user's prescribed series of steps associated with that user's life sciences research.

41. A life sciences network portal system comprising:
at least one networked computer system that defines a portal through which a user may access said networked computer system to conduct life sciences research, the networked computer system including at least one processor that is configured to perform bioinformatics processing upon user-specified data;
a workflow system associated with said portal and operable to allow a user to prescribe and track the performance of a series of steps associated with the user's life sciences research;
a data store of life sciences information accessible through said portal into which said user stores and organizes at least one set of user-specified data for bioinformatics processing;
a product specifying system accessible through said portal and offering products useful in connection with the performance of said series of steps associated with the user's life sciences research; and
an indexing mechanism associated with said networked computer system that mediates relationships among said workflow system, said data store of life sciences information and said product specifying system;
wherein said workflow system allows the user to manipulate user-specified data stored in said data store and whereby said user can control the performance of said bioinformatics processing.

42. The system of claim 41, further comprising a purchasing subsystem adapted to communicate life sciences related consumptibles to the user, receive a user selection of a consumptible, and process a purchase of the consumptible by the user based on the user selection; and
wherein said indexing mechanism further mediates relationships among said workflow system, said data store of life sciences information, said product specifying system, and said purchasing subsystem.

43. The system of claim 41, wherein the indexing mechanism is synchronized in real-time with the product specifying system.
